SEQUENCE LISTING (2 sequences)

SEQ ID NO: 1 - DNA, 21 bp, artificial sequence
  primer
atggtgaaggccgtcgccgtc

SEQ ID NO: 2 - DNA, 21 bp, artificial sequence
  primer
ttaaccctggaggccaataat

(19) United States
(12) Patent Application Publication
(10) Pub. No.: US 2009/0170166 A1
(43) Pub. Date: Jul. 2, 2009

(54) ANTIBODY INHIBITING URACIL-DNA-GLYCOSYLASE AND USES THEREOF FOR DECONTAMINATING NUCLEIC ACID AMPLIFICATION REACTIONS

(76) Inventors: Alphonse Calenda, Angers (FR); Bernard Montanari, Paris (FR); Bruno Montanari, Paris (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/596,965

(22) PCT Filed: Dec. 22, 2004
(86) PCT No.: PCT/FR2004/003336

§ 371 Date: Jun. 10, 2008

(30) Foreign Application Priority Data
Dec. 31, 2003 (FR) 0315625

(51) Int. Cl. C12P 19/34; C07K 16/18

(52) U.S. Cl. 435/91.2; 530/387.9

(57) ABSTRACT

The invention concerns antibodies directed against the uracil-DNA-glycosylase inhibitor, and uses thereof for decontaminating nucleic acid amplification reactions.

The present invention relates to antibodies directed against the uracil-DNA-glycosylase inhibitor, and to their applications for the decontamination of nucleic acid amplification reactions.

Methods for amplifying nucleic acids such as the polymerase chain reaction (PCR) are commonly used in numerous fields for the detection of nucleic acids in samples of diverse origins or the preparation of nucleic acids. However, because of the sensitivity of these methods which make it possible to amplify minute quantities of target nucleic acid, the amplification reactions can be contaminated with nucleic acid generated during previous amplification reactions, present in the reagents, the material or the work environment. This contaminating nucleic acid, which is amplified during subsequent PCR reactions, is at the origin of false-positive results.

To inactivate this contaminating nucleic acid, it has been proposed to incorporate, into the amplified sequences, deoxyuridine triphosphate nucleotides (dUTP) in place of deoxythymidine triphosphate nucleotides (dTTP). The contaminating DNA, which contains deoxyuridine, is then specifically removed with the enzyme uracil-DNA-glycosylase (UDG for Uracil-DNA-Glycosylase or UNG for Uracil-N-Glycosylase) which cleaves the uracil residue, thus generating apyrimidine sites which block the amplification of the contaminating DNA. More specifically, prior to the amplification reaction, the sample is treated with UDG so as to remove the contaminating DNA, and then the UDG is heat inactivated so as to avoid the destruction of the newly amplified DNA (American patent U.S. Pat. No. 5,418,149 in the name of HOFFMANN-LA ROCHE INC; American patent U.S. Pat. No. 5,035,996 and European patents EP0401037 and EP0415755, in the name of LIFE TECHNOLOGIES INC).

However, it has been shown that the heat treatment induces partial inactivation of the UDG which leads to the destruction of the newly synthesized DNA molecules. Indeed, UDG is inactivated during the amplification reaction which is carried out at high temperatures but a uracil-DNA-glycosylase activity, sufficient to degrade the newly amplified DNA, is detected at the end of the amplification reaction, when the samples are placed at room temperature or at a lower temperature (about +4° C. to +25° C.).

To inhibit the residual UDG activity present at the end of the PCR reaction, it has been proposed to use either thermolabile UDG (International application PCT WO9201814 in the name of CETUS CORPORATION), or a UDG inhibitor (Ugi for Uracil-DNA-glycosylase inhibitor; Thornton et al., Biotechniques, 1992, 13:180-184, American patent U.S. Pat. No. 5,536,649 in the name of BECTON, DICKINSON AND COMPANY).

However, the use of Ugi is cumbersome to carry out since the addition of this inhibitor, either at the end of the step for decontamination with UDG, or at the end of the amplification reaction, involves additional handling of the devices (tubes, plates) containing the reaction mixtures.

Consequently, the inventors set themselves the aim of developing a method for decontaminating nucleic acid amplification reactions which is effective and simple to use and which thus better satisfies the requirements for practical use than the prior art methods.

The inventors prepared anti-Ugi antibodies and they showed that the use of Ugi/anti-Ugi antibody complexes, in place of Ugi, advantageously made it possible to carry out decontamination and amplification of DNA, without additional handling; the starting reaction mixture containing, in addition to the sample containing the nucleic acids to be amplified and the reagents essential for the amplification of these nucleic acids, UDG and inactivated Ugi, in the form of reversible Ugi/anti-Ugi antibody complexes.

Consequently, the subject of the present invention is an antibody or a functional fragment of an antibody comprising at least the variable domains of the heavy and light chains, characterized in that it binds specifically to the uracil-DNA-glycosylase inhibitor (Ugi) of the sequence SWISSPROT P14739 and in that it inhibits the binding between uracil-DNA-glycosylase (UDG) and its inhibitor, Ugi.

The anti-Ugi antibody and antibody fragment according to the present invention are Ugi antagonists possessing the following properties:
they bind specifically to Ugi and possess a high affinity for Ugi, and

they are capable of inhibiting the binding between UDG and Ugi.

These properties can be verified by conventional tests which make it possible to evaluate the binding activity of an antibody to a protein or to a peptide or the inhibition of this activity, in particular by direct and competition ELISA tests. For example, the ELISA titer of the anti-Ugi polyclonal antibodies, with regard to purified recombinant Ugi, is high (more than 1/10⁶).

In accordance with the invention, said antibody is a monoclonal antibody or a polyclonal antibody and said antibody fragment is an Fab or an Fv or an scFv.

The antibody or antibody fragment according to the invention are prepared by conventional techniques known to a person skilled in the art, such as those described in Antibodies: A Laboratory Manual, E. Howell and D. Lane, Cold Spring Harbor Laboratory, 1988.

More specifically:
the uracil-DNA-glycosylase inhibitor is produced in E. coli from an appropriate expression vector, it is then purified, as described in Wang et al. (Genetics 1991, 99, 31-37), the peptide fragments of Ugi are produced by conventional techniques for peptide synthesis or for expression of recombinant DNA.
the polyclonal antibodies are prepared by immunizing an appropriate animal with the uracil-DNA-glycosylase inhibitor or one of its fragments, optionally coupled to KLH or to albumin and/or combined with an appropriate adjuvant such as Freund's adjuvant (complete or incomplete) or aluminum hydroxide; after obtaining a satisfactory antibody titer, the antibodies are harvested by collecting serum from the immunized animals and enriched in IgG by precipitation, according to conventional methods, and then the IgGs specific for Ugi are optionally purified by affinity chromatography on an appropriate column to which Ugi or one of its fragments is attached, so as to obtain a monospecific IgG preparation.
the monoclonal antibodies are produced from hybridomas obtained by fusion of B lymphocytes of an animal immunized with myelomas, according to the Köhler and Milstein technique (Nature, 1975, 256, 495-497); the hybridomas are cultured in vitro, in particular in fermenters or produced in vivo, in the form of ascites.
the antibody fragments are produced from cloned V_{H} and V_{L} regions, from mRNAs of hybridomas or spleen lymphocytes of an immunized animal; for example, the Fv, scFv or Fab fragments are expressed at the surface of filamentous phages according to the Winter and Milstein technique (Nature, 1991, 349, 293-299); after several selection steps, the phages which express the antibody fragments specific for the antigen are isolated and the cDNAs corresponding to said fragments are expressed in an appropriate expression system, by conventional techniques of cloning and expression of recombinant DNA.

The monoclonal and polyclonal antibodies or fragments thereof as defined above are purified by conventional techniques known to persons skilled in the art, in particular by affinity chromatography.

According to an advantageous embodiment of said antibody, it is a polyclonal antibody obtained by immunizing an animal with a preparation of recombinant uracil-DNA-glycosylase inhibitor.

The subject of the present invention is also the use of an anti-Ugi antibody as defined above, as antagonist for the binding between Ugi and UDG.

According to an advantageous embodiment of the invention, said antibodies are used to decontaminate nucleic acid amplification reactions, in particular polymerase chain reactions (PCR).

The subject of the present invention is additionally a method for amplifying decontaminated nucleic acids, comprising the following steps:

a) incubation of a reaction mixture containing: a nucleic acid sample to be amplified, the reagents necessary for its amplification including deoxyuridine triphosphate (dUTP) nucleotides, uracil-DNA-glycosylase (UDG), uracil-DNA-glycosylase inhibitor (Ugi), and an anti-Ugi antibody, at a temperature of between 25° C. and 60° C., preferably at 37° C., for a sufficient time to allow deglycosylation of the nucleic acids containing deoxyuridine, and
b) incubation of said mixture at a temperature of between 60° C. and 98° C., preferably between 90° C. and 98° C., for a sufficient time to allow denaturation of the anti-Ugi antibody and release of Ugi, and
c) amplification of the DNA under appropriate conditions.

In accordance with the invention:
said reaction mixture of step a) comprises the nucleic acid sample to be amplified (DNA or cDNA), nucleotides dATP, dCTP, dGTP, dUTP, oligonucleotide primers, Mg²⁺ ions and a DNA polymerase, in an appropriate buffer,
the duration of the incubation of step a) varies according to the quantity of contaminating DNA (DNA containing deoxyuridine) to be degraded with uracil-DNA-glycosylase and may be determined experimentally; it is generally less than one hour, preferably from 30 s to 30 min, preferably from 5 min to 10 min,
the duration of the incubation of step b) is generally less than one hour, preferably from 30 s to 30 min, preferably from 5 min to 10 min.

The method according to the invention, which comprises the digestion of the contaminating DNA with UDG (step a) and then the release of the Ugi (step b) from a starting reaction mixture, makes it possible to decontaminate the DNA without destroying the newly synthesized DNA and does not involve an additional step of adding Ugi, either at the end of the decontamination step, or at the end of the amplification reaction.

More specifically:
in step a), the active UDG destroys the contaminating nucleic acids containing dUTP while the Ugi is inactivated in the form of reversible Ugi/anti-Ugi antibody complexes,
in step b), heat inactivates the UDG, dissociates the Ugi-antibody complexes and denatures the antibodies irreversibly, while the Ugi is released,
in step c), the Ugi binds to the UDG in the form of UDG-Ugi complexes and inhibits the activity of UDG during the subsequent DNA amplification steps.

For the purposes of the present invention, the expression reversible Ugi-anti-Ugi antibody complex is understood to mean a complex that is stable in the presence of UDG but which is dissociated by the action of heat which irreversibly denatures the anti-Ugi antibodies.

The subject of the present invention is additionally a kit for decontaminating nucleic acid amplification reactions, characterized in that it comprises at least one antibody or one antibody fragment according to the present invention, preferably in the form of reversible Ugi-antibody or Ugi-antibody fragment complexes.

In addition to the preceding features, the invention also comprises other features which will emerge from the description which follows, which refers to examples of preparation of anti-Ugi antibodies which are the subject of the present invention, and the use of these antibodies for the decontamination of nucleic acid amplification reactions, and the accompanying drawings in which:

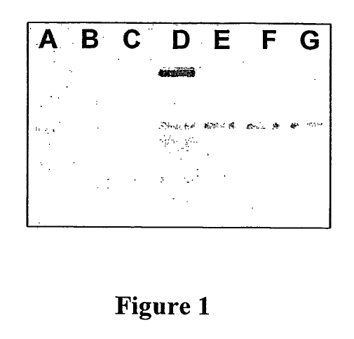

FIG. 1 illustrates the optimization of the UDG concentration in the reaction mixture; a large excess of contaminating DNA (100 ng) containing dUTP or dTTP (control) is digested for one hour at 37° C. in a reaction mixture containing UDG diluted 1/25 or 1/50, corresponding to the final concentrations of 0.04 U/μl and 0.02 U/μl, respectively. A: DNA dUTP. B: DNA dUTP+UDG 1/25. C: DNA dUTP+UDG 1/50. D: molecular weight marker. E: DNA dTTP. F: DNA dTTP+UDG 1/25. G: DNA dTTP+UDG 1/50.

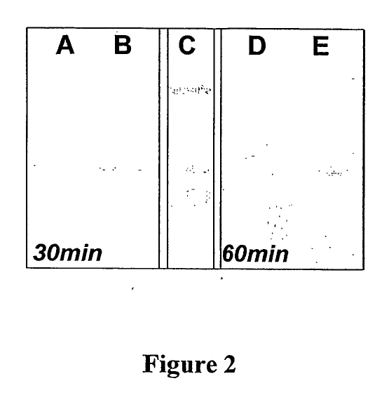

FIG. 2 illustrates the optimization of the duration of the digestion of the contaminating DNA with UDG; a large excess of contaminating DNA (100 ng) containing dUTP was digested for 30 min (A and B) or one hour (D and E) at 37° C. in a reaction mixture without UDG (B and E) or containing UDG diluted 1/25 (0.04 U/μl: A and D).

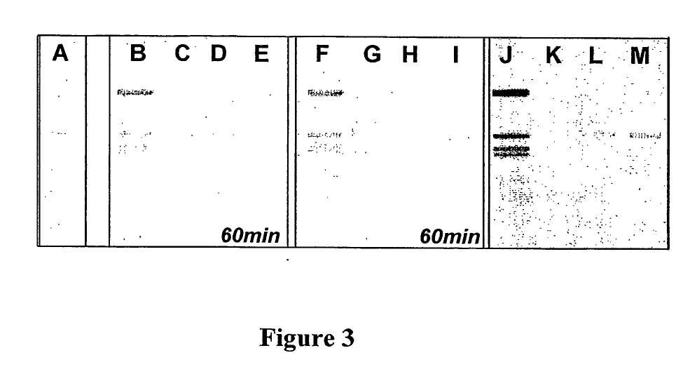

FIG. 3 illustrates the optimization of the concentration of Ugi which makes it possible to inhibit the digestion of contaminating DNA with UDG; a large excess of contaminating DNA (100 ng) containing dUTP was digested for 60 min at 37° C., in the presence of UDG diluted 1/25 or 1/50 and of Ugi diluted 1/20 000 or 1/30 000 (C, D, E, G, H, I), or for 90 min at 37° C., in the presence of UDG diluted 1/50 and of Ugi diluted 1/20 000 or 1/30 000 (K, L, M). A: DNA dUTP. B, F and J: molecular weight marker. C: DNA dUTP+UDG 1/25. G and K: DNA dUTP+UDG 1/50. D: DNA dUTP+UDG 1/25. G Ugi 1/20 000. E: DNA dUTP+UDG 1/25+Ugi 1/30 000. H and L: DNA dUTP+UDG 1/50+Ugi 1/20 000. I and M: DNA dUTP+UDG 1/50+Ugi 1/30 000.

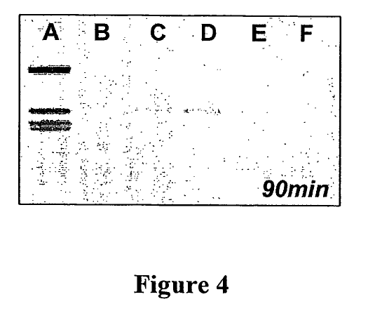

FIG. 4 illustrates the optimization of the anti-Ugi immune serum (I.S.) concentration which makes it possible to antagonize the inhibitory effect of Ugi on the digestion of the contaminating DNA with UDG; the preimmune serum (P.I.) is used as a control. The 460 bp DNA amplified in the presence of dUTP (DNA-dUTP) was digested for 90 min at 37° C., in the presence of UDG diluted 1/50, of Ugi diluted 1/20 000 or 1/30 000, and of anti-Ugi immune serum diluted 1/100 or of preimmune serum diluted 1/100. A: molecular weight marker. B: DNA-dUTP+UDG 1/50. C: DNA-dUTP+UDG 1/50+Ugi 1/20 000+P.I. 1/100. D: DNA-dUTP+UDG 1/50+Ugi 1/30 000+P.I. 1/100. E: DNA-dUTP+UDG 1/50+Ugi 1/20 000+I.S. 1/100. F: DNA-dUTP+UDG 1/50+Ugi 1/30 000+I.S. 1/100.

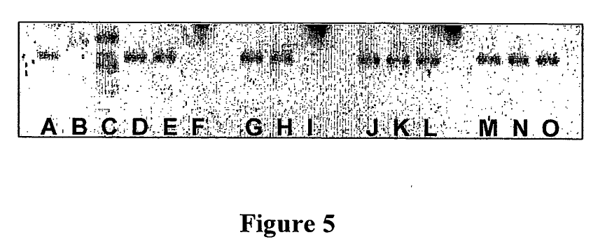

FIG. 5 illustrates the optimization of the conditions for denaturing the anti-Ugi antibodies; two identical series of 460 bp DNA amplified in the presence of dUTP (DNA-dUTP) are exposed to UDG diluted 1/35, to Ugi diluted 1/20 000 or 1/30 000, and to anti-Ugi immune serum diluted 1/100 or to preimmune serum diluted 1/100. Prior to the digestion step of 60 minutes at 37° C., one of the two series is preincubated for 10 min at 90° C. (J, K, L, M, N and O). A: DNA-dUTP. B: DNA-dUTP+UDG 1/35. C: molecular weight marker. D and J: DNA-dUTP+UDG 1/35+Ugi 1/20 000. E and K: DNA-dUTP+UDG 1/35+Ugi 1/20 000+P.I. 1/100. F and L: DNA-dUTP+UDG 1/35+Ugi 1/20 000+I.S. 1/100. G and M: DNA-dUTP+UDG 1/35+Ugi 1/30 000. H and N: DNA-dUTP+UDG 1/35+Ugi 1/30 000+P.I. 1/100. I and O: DNA-dUTP+UDG 1/35+Ugi 1/30 000+I.S. 1/100.

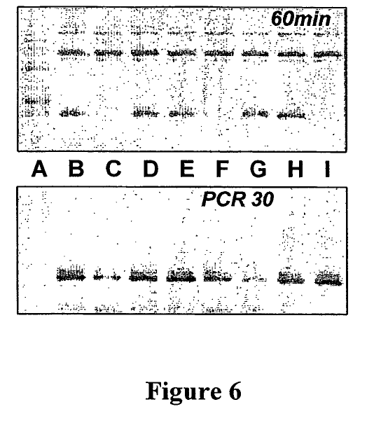

FIG. 6 illustrates the PCR amplification of the template DNA under the decontamination conditions established in Example 2; the contaminating DNA (DNA-dUTP) is mixed with the template plasmid DNA (DNA containing dTTP), UDG 1/35, in combination or otherwise with Ugi (1/20 000 or 1/30 000) and with the anti-Ugi immune serum (I.S.) or the preimmune serum (P.I., control), and amplified under conditions as defined in Example 2. A: molecular weight marker. B: DNA-dUTP. C: DNA-dUTP+UDG 1/35. D: DNA-dUTP+UDG 1/35+Ugi 1/20 000. E: DNA-dUTP+UDG 1/35+Ugi 1/20 000+P.I. 1/100. F: DNA-dUTP+UDG 1/35+Ugi 1/20 000+I.S. 1/100. G: DNA-dUTP+UDG 1/35+Ugi 1/30 000. H: DNA-dUTP+UDG 1/35+Ugi 1/30 000+P.I. 1/100. I: DNA-dUTP+UDG 1/35+Ugi 1/30 000+P.I. 1/100. Top plate: incubation 60 minutes at 37° C. Bottom plate: incubation 60 minutes at 37° C. and PCR amplification under the conditions as defined in Example 2.

### EXAMPLE 1

### Preparation and Characterization of Polyclonal Antibodies Directed Against Ugi

1) Preparation of Recombinant Ugi (rUgi)

Overlapping oligonucleotides representing the complete coding sequence of Ugi (Wang and Mosbaugh, J. Biol. Chem., 1989, 264, 1163-) were hybridized with their complementary sequence, and then extended and amplified by the polymerase chain reaction (PCR). The amplification product obtained was digested with appropriate restriction enzymes, cloned into the vector pUC18 (PHARMACIA) and then the conformity of the sequence inserted into the recombinant plasmid was verified by automated sequencing. A fragment of the recombinant plasmid pUC18 corresponding to the sequence encoding Ugi was then cloned into the expression vector pQE80L (QIAGEN). The E. coli DH5α strain was transformed with the recombinant expression vector thus obtained and the recombinant Ugi protein produced by the transformed bacteria was purified on an Ni-NTA agarose support according to the recommendations of the manufacturer (QIAGEN). More specifically, the recombinant Ugi retained on the Ni-NTA support was eluted in the presence of 100 mM or 250 mM of imidazole and then the imidazole present was removed by ultrafiltration.

Analysis of the protein preparation obtained by polyacrylamide gel electrophoresis under denaturing conditions followed by staining with Coomassie blue shows that the protocol used makes it possible to purify the Ugi protein with a very satisfactory homogeneity and an average yield of 10 mg of protein per liter of culture.

### 2) Preparation of an Anti-rUgi Polyclonal Immune Serum

2.1) Coupling of rUgi to KLH

The purified recombinant Ugi protein (rUgi) prepared according to the protocol described in the preceding paragraph was coupled to KLH (Keyhole Limpet Hemocyanin, PIERCE), with the aid of glutaraldehyde (SIGMA), following the coupling protocol described in Habeeb and Hiramoto et al. (Arch. Biochem. Biophys., 1998, 126, 6-). Alternatively, the recombinant Ugi protein is that available from a supplier such as NEW ENGLAND BIOLABS (reference M0281). The KLH-coupled rUgi was then purified and the rUgi/KLH molecular ratio was calculated according to the protocol described in Briand et al., Immunol. Methods, 1985, 93, 9-). The preparation of rUgi is estimated at about 500 molecules of rUgi per molecule of KLH.

### 2.2) Immunization of Rabbits

Rabbits were immunized with rUgi coupled to the KLH prepared according to the protocol described in the preceding paragraph. After a first multipoint intradermal injection of 0.15 mg per rabbit, of coupled protein, emulsified in complete Freund's adjuvant, the animals received two booster injections, at an interval of three weeks, of 0.025 mg of coupled protein, emulsified in incomplete Freund's adjuvant.

For each rabbit, a preimmune serum (P.I.) was prepared before the first immunization and an immune serum (I.S.) one week after the last immunization.

### 3) Analysis of the ELISA Reactivity of an Anti-Ugi Polyclonal Serum

The reactivity of the sera was analyzed by an ELISA test, with regard to a recombinant protein preparation similar to that used for the immunizations.

The recombinant Ugi protein diluted 1/1000 in 0.1 M sodium phosphate buffer, pH 7.4, is distributed in an amount of 100 μl (0.1 μg) in the wells of ELISA plates, and then the plates are incubated overnight at laboratory temperature. The plates are washed with PBS-Tween 20 buffer (0.1%), saturated with PBS buffer-bovine serum albumin Fraction V (BSA: 1%). The sera to be tested (100 μl), diluted beforehand, are added and then the plates are incubated for 1 h at 37° C. After 3 washings, the anti-rabbit IgG conjugate labeled with peroxidase is added to the dilution recommended by the manufacturer and then the plates are incubated for 1 h at 37° C. After 4 washings, the chromogen (ortho-phenylenediamine, OPD) and the substrate (H₂O₂, 110 volumes) are added and the plates are incubated for 20 min at room temperature, protected from light. The reaction is then stopped by the addition of sulfuric acid (H₂SO₄, 12.5%) and then the absorbance at 492 nm is measured with the aid of an automated reader.

The results of the ELISA tests demonstrate that the recombinant Ugi protein preparation is immunogenic in animals and that the titer of the immune sera is high (over 1/10⁶).

### EXAMPLE 2

### Optimization of the Decontamination Conditions in the Presence of Ugi-Anti-Ugi Antibody Complexes

### 1) Materials and Methods

### a) Primers/DNA Template

The primers CytTo5′ (atggtgaaggccgtcgccgtc, SEQ ID NO. 1) and CytTo3′ (ttaaccctggaggccaataat, SEQ ID NO. 2) are used to amplify a fragment of 460 bp corresponding to the cDNA encoding the tomato copper-zinc cytosolic SOD (GENBANK X1040 and Perl-Treves, R. et al., Plant Mol. Biol., 1988, 11, 609-623), from a recombinant plasmid containing said cDNA, used as template for the polymerase chain reaction (PCR).

### b) Amplification Reaction (PCR)

The reaction mixture contains, in a volume of 50 μl of PCR buffer: DNA template (25 ng), primers (0.2 μM), MgCl₂ (1.5 mM), each of the dNTPs (200 μM) in the form of a mixture including dTTP (control) or dUTP, and DNA polymerase (Taq, 2.5 IU).

The amplification is carried out under the following conditions: an initial step of denaturation at 95° C. for 5 min is followed by 30 cycles comprising: a step of denaturation at 94° C. for 40 sec, a step of annealing at 58° C. for 40 sec and then a step of extension at 72° C. for 50 sec, and then a final step of extension at 72° C. for 10 min. The amplification products obtained are stored at +4° C. before being analyzed by agarose gel electrophoresis (1.2% in TAE buffer), in the presence of ethidium bromide.

### c) Other Reagents

Contaminating DNA

The contaminating DNA consists of an amplification product as above, obtained in the presence of dUTP; it is used in a large excess (100 ng), so as to be able to be visualized on an agarose gel stained with ethidium bromide. The DNA amplified in a similar manner, but in the presence of dTTP instead of dUTP, is used as a control for the specificity of the decontamination.

UDG

The UDG (LIFE TECHNOLOGIES; 1 IU/μl) is used at the dilutions of 1/25, 1/35 and 1/50 corresponding respectively to the following final concentrations: 0.04 U/μl, 0.028 U/μl and 0.02 U/11.

rUgi

The rUgi prepared as described in Example 1 (1.1 mg/ml) is used at the dilutions of 1/20 000, 1/30 000 corresponding respectively to the following final concentrations: 55 ng/ml and 36 ng/ml.

Antibody

The anti-Ugi immune serum and the preimmune serum serving as a control, prepared as described in Example 1, are used at 1/100.

2) Determination of the UDG Concentration and of the Duration of Digestion of the Contaminating DNA with UDG

After having verified that the 460 bp DNA was amplified in a similar manner, in the presence of dTTP or of dUTP, the amplification products thus obtained, called (DNA-dTTP and DNA-dUTP; 100 ng), were digested for one hour at 37° C., in the presence of UDG diluted 1/25 or 1/50 (FIG. 1), or for 30 min or one hour at 37° C., in the presence of UDG diluted 1/25 (FIG. 2).

FIGS. 1 and 2 show that a large excess of contaminating DNA (100 ng) is completely and specifically digested by incubation for one hour at 37° C., in the presence of UDG diluted 1/25 (0.04 U/μl).

3) Determination of the Ugi Concentration which Makes it Possible to Inhibit the Digestion of Contaminating DNA with UDG

The 460 bp DNA amplified in the presence of dUTP (DNA-dUTP) was digested for 60 min at 37° C., in the presence of UDG diluted 1/25 or 1/50 and of Ugi diluted 1/20 000 or 1/30 000, or for 90 min at 37° C., in the presence of UDG diluted 1/50 and Ugi diluted 1/20 000 or 1/30 000 (FIG. 3).

FIG. 3 shows that at the highest concentration of UDG, the UDG activity may be inhibited by Ugi at 1/20 000 but not at 1/30 000. On the other hand, at the lowest concentration of UDG, the UDG activity is inhibited both by Ugi at 1/20 000 and at 1/30 000.

4) Determination of the Concentration of Anti-Ugi Immune Serum which Makes it Possible to Antagonize the Inhibitory Effect of Ugi on the Digestion of the Contaminating DNA by UDG

The 460 bp DNA amplified in the presence of dUTP (DNA-dUTP), was digested for 90 min at 37° C., in the presence of UDG diluted 1/50, Ugi diluted 1/20 000 or 1/30 000, and of anti-Ugi immune serum diluted 1/100 or of preimmune serum diluted 1/100.

FIG. 4 shows that at the two Ugi concentrations tested, the anti-Ugi serum is capable of antagonizing the inhibitory effect of Ugi on UDG, thus allowing UDG to completely digest the contaminating DNA.

### 5) Determination of the Conditions for Denaturation of the Anti-Ugi Antibodies

The 460 bp DNA amplified in the presence of dUTP (DNA-dUTP) is preincubated or otherwise for 10 minutes at 90° C., in the presence of UDG diluted 1/35, of Ugi diluted 1/20 000 or 1/30 000, and of anti-Ugi immune serum diluted 1/100 or of preimmune serum diluted 1/100, and then the various mixtures are digested for 60 min at 37° C.

FIG. 5 shows that under these conditions:
the contaminating DNA (DNA-dUTP) is indeed digested by UDG (lane B),
the two dilutions of Ugi indeed inhibit the digestion of DNA by UDG (lanes D and G) and that this inhibition is antagonized by the anti-Ugi immune serum (lanes F and I),
after a treatment of 10 minutes at 90° C., the Ugi remains active because it still inhibits the UDG (lanes J and M) and therefore protects the contaminating DNA from digestion by UDG,
after this same treatment, the antibody was denatured and it is then incapable of antagonizing the inhibitory effect of Ugi (lanes L and O),
after this same treatment, the Ugi thus released then becomes capable of inhibiting the UDG and prevents the degradation of the DNA-dUTP.

### 6) Amplification of DNA Decontaminated Under the Decontamination Conditions Thus Determined

The contaminating DNA (DNA-dUTP) is mixed with the template plasmid DNA, the UDG (1/35), in combination or otherwise with Ugi (1/20 000 or 1/30 000) and the anti-Ugi immune serum (I.S., 1/100) or the preimmune serum (P.I., control, 1/100) and amplified under the conditions as defined in paragraph 1.

FIG. 6 shows that:
after an incubation of 60 minutes at 37° C., the UDG at 1/35 indeed digests the DNA dUTP but not the template DNA which contains dTTP (lane C),
the two dilutions of Ugi indeed inhibit UDG (lanes D and G) and the preimmune serum has no effect on this inhibition (lanes E and H),
the anti-Ugi antibody antagonizes the inhibitory effect of Ugi on UDG, thus allowing UDG to completely digest the contaminating DNA (lanes F and I),
the various constituents of the reaction mixture which make it possible to degrade the contaminating DNA from a single reaction mixture, do not interfere with the amplification of the template DNA after 30 amplification cycles or with the integrity of the amplification product obtained (no degradation of the neosynthesized DNA-dUTP).

1. An antibody or a functional fragment of an antibody comprising at least the variable domains of the heavy and light chains, characterized in that it binds specifically to the uracil-DNA-glycosylase inhibitor (Ugi) of the sequence SWISSPROT P14739 and in that it inhibits the binding between uracil-DNA-glycosylase (UDG) and its inhibitor, Ugi.

2. The antibody or antibody fragment as claimed in claim 1, characterized in that it is chosen from monoclonal antibodies, polyclonal antibodies and the Fab, Fv and scFv fragments.

3. The antibody as claimed in claim 2, characterized in that it is a polyclonal antibody obtained by immunizing an animal with a preparation of recombinant uracil-DNA-glycosylase inhibitor.

4. The use of an antibody or an antibody fragment as claimed in claim 1, as antagonist of the binding between uracil-DNA-glycosylase and its inhibitor.

5. The use as claimed in claim 4, for decontaminating nucleic acid amplification reactions, in particular polymerase chain reactions.

6. A method for amplifying decontaminated nucleic acids, characterized in that it comprises the following steps:
a) incubation of a reaction mixture containing: a nucleic acid sample to be amplified, the reagents necessary for its amplification including deoxyuridine triphosphate nucleotides, uracil-DNA-glycosylase, uracil-DNA-glycosylase inhibitor, and an anti-uracil-DNA-glycosylase inhibitor antibody or antibody fragment as claimed in claim 1, at a temperature of between 25° C. and 60° C., for a sufficient time to allow the deglycosylation of the nucleic acids containing deoxyuridine, and
b) incubation of said mixture at a temperature of between 60° C. and 98° C., for a sufficient time to allow the denaturation of the anti-uracil-DNA-glycosylase inhibitor antibody and the release of Ugi, and
c) amplification of the DNA under appropriate conditions.

7. The method as claimed in claim 6, characterized in that the incubation in steps a) and b) is carried out for less than one hour.

8. The method as claimed in claim 6, characterized in that the anti-uracil-DNA-glycosylase inhibitor antibody and the uracil-DNA-glycosylase inhibitor form a reversible complex.

9. A kit for decontaminating nucleic acid amplification reactions, characterized in that it comprises at least one antibody or an antibody fragment as claimed in claim 1 in the form of a reversible complex with the uracil-DNA-glycosylase inhibitor.
